(19) United States
(12) Patent Application Publication
(10) Pub. No.: US 2022/0346462 A1
(43) Pub. Date: Nov. 3, 2022

(54) Mobility Support Sock

(71) Applicant: KaNisha L. Hall, Houston, TX (US)

(72) Inventor: KaNisha L. Hall, Houston, TX (US)

(21) Appl. No.: 17/244,892

(22) Filed: Apr. 29, 2021

(51) Int. Cl. A41B 11/04; A61F 13/08; A61F 13/06

(52) U.S. Cl. CPC A41B 11/04; A61F 13/085; A61F 13/062; A41B 11/01

(57) ABSTRACT

A sock having a femur section and a patella section. The patella section has an outer layer, an inner layer, and a slit on the outer layer. The sock has a compressed tibia section, and a compression strap positioned between the outer layer and the inner layer. The compression strap has a first end extending through the slit and a second end extending through the slit.

### BACKGROUND

The mobility and strength of our lower extremities tend to diminish as we age. Particularly joints, such as our knees, are comprised of bones, muscles, ligaments, arteries, and veins that all individually and in concert serve special functions towards our ability to walk or run. The deterioration of our lower extremities, whether through lack of blood circulation, stiffness of the lateral and medial collateral ligaments, injury, or age often require invasive corrective surgery. Invasive corrective surgery is not an option for many. Therefore, providing continuous support to an underperforming joint or group of muscles without invasive surgery is a challenge.

### BRIEF DESCRIPTION OF THE DRAWINGS

FIG. 1 is a rear view of a leg wearing a mobility support sock.

FIG. 2 is a profile view of a leg wearing a mobility support sock.

FIG. 3 is a frontal view of a leg wearing a mobility support sock.

FIG. 4 is a superior view of a patella section of a leg wearing a mobility support sock.

FIG. 5 is an illustration of a compression strap having Velcro straps.

FIG. 6 is an illustration of a compression strap having a buckle connection.

FIG. 7 is an illustration of a compression strap having a double ring strap.

FIG. 8 is a flow chart illustrating the method of using a mobility support sock.

### DETAILED DESCRIPTION

The following detailed description illustrates embodiments of the present disclosure. These embodiments are described in sufficient detail to enable a person of ordinary skill in the art to practice these embodiments without undue experimentation. It should be understood, however, that the embodiments and examples described herein are given by way of illustration only, and not by way of limitation. Various substitutions, modifications, additions, and rearrangements may be made that remain potential applications of the disclosed techniques. Therefore, the description that follows is not to be taken as limiting on the scope of the appended claims. In particular, an element associated with a particular embodiment should not be limited to association with that particular embodiment but should be assumed to be capable of association with any embodiment discussed herein.

The mobility and strength of our lower extremities tend to diminish as we age. Particularly joints, such as our knees, are comprised of bones, muscles, ligaments, arteries, and veins that all serve special functions in our ability to walk or run. For example, the knee is a joint that is comprised of bones, muscles, and ligaments. The bones provide structure, shape, and support, whereas the muscles provide strength and movement. The ligaments are the connective tissue components that stabilizes the knee and allow the bones and joints to move without collapsing or giving out. As individuals age, the ligaments, such as the lateral and medial collateral ligaments become stiff and weak. These changes make the joints more susceptible to injury. Normal aging may contribute to decreased blood circulation in the leg, which can result in further joint deterioration or worse, such as blood clots. The deterioration of our legs, whether through lack of blood circulation, stiffness of the lateral and medial collateral ligaments, injury, or age may often result in the need for invasive corrective surgery. Invasive surgery at any age is risky, but especially risky for those who are aging. The embodiments described herein provides non-surgical options to individuals by providing a mobility support sock with support straps and compression material that aid in compensating reduced strength, compromise blood flow, and stiff ligaments of the lower extremities. In addition, this device provides stability and support to maintain mobility, which could reduce the need for invasive surgery.

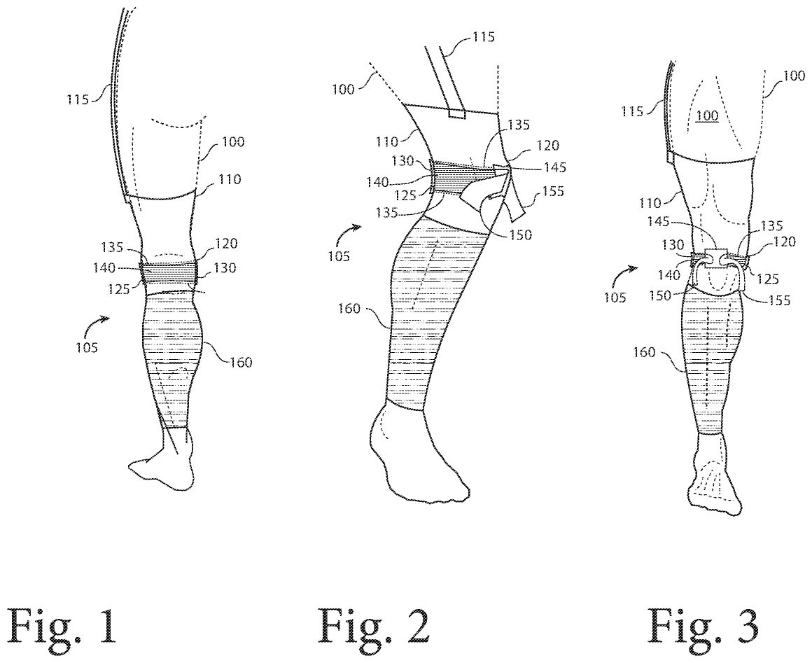

FIG. 1 is a rear view of a leg wearing a mobility support sock. FIG. 2 is a profile view of a leg wearing a mobility support sock. FIG. 3 is a frontal view of a leg wearing a mobility support sock. As illustrated in FIGS. 1-3, the embodiments to support a leg 100 of a person includes a mobility sock 105. The mobility sock 105 may be made of nylon, cotton, polyurethane, or other stretchable synthetic material. The mobility sock 105 may be manufactured in varying sizes to include foot sizes ranging from shoe size five (i.e., nine inches) to shoe size 25 (i.e., 15.66 inches) in men; and shoe size three (eight inches) to shoe size 20 (13.66 inches) in women. The height of the sock from the base of the foot may vary in size and may include a height ranging between 15 inches and 30 inches.

In one or more embodiments, the mobility sock 105 includes a femur section 110. The femur section 110 is the section of the leg 100 commonly known as the thigh. The femur section 110 of the mobility sock 105 may have a height of between three inches to six inches. The femur section may include compression material that compresses the femur section of the leg 100. The compression rate of the compressed material may have a range of between five to thirty mmHg (i.e, millimeters of mercury). The femur section 110 may include a suspender strap 115. The suspender strap 115 is used to provide additional support for the mobility sock 105 such that the mobility sock 105 does not slide down the individual's leg 100 while in operation. The suspender strap 115 is coupled to the femur section 110 by a clamp, snap button, or other similar coupling apparatus. The other end of the suspender strap 115 (i.e., opposite the femur section 110) is coupled to the individual's briefs, undergarment, or other similar undergarments (not illustrated).

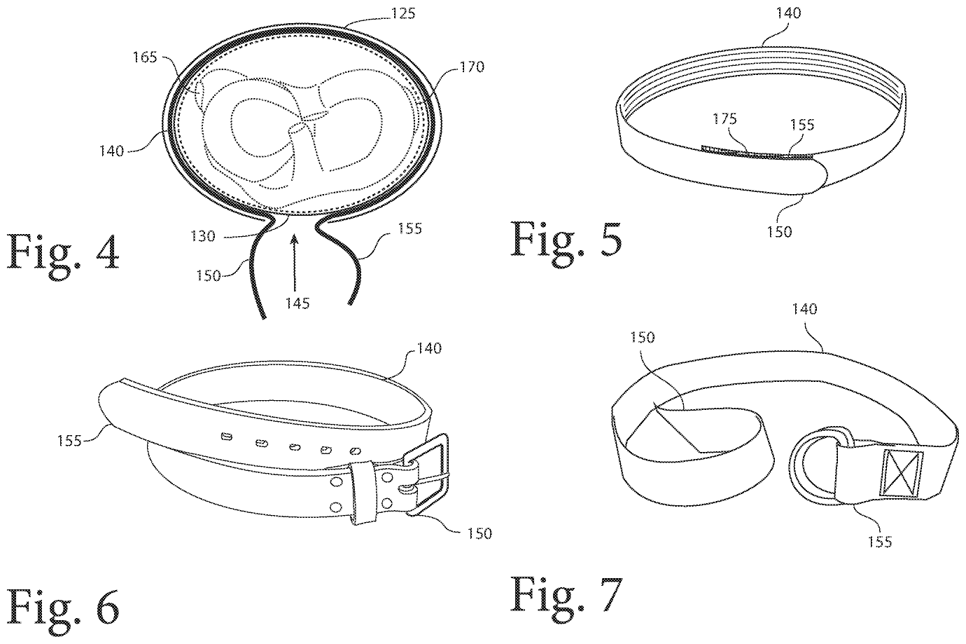

The mobility sock 105 may include a patella section 120. The patella section 120 is the section of the leg 100 commonly known as the knee. The patella section 120 may have a height ranging between two inches to four inches. In one or more embodiments, as illustrated in FIGS. 1-3 (and more clearly illustrated in FIG. 4) the patella section 120 may have an outer layer 125 and an inner layer 130 creating a passage between the two layers. Securing the two layers (i.e., outer layer 125 and inner layer 130) together is a slip seam 135. The slip seam 135 is the thread that couples the inner layer 130 and outer layer 125 together forming the passage. The slip seam 135 also serves as a guide to limit vertical movement of a compression strap 140.

The compression strap 140 may be positioned between the inner layer 130 and outer layer 125 and extending through a slit 145 located at the patella section 120 as illustrated in FIGS. 2 and 3. The slit 145 is an opening on the outer layer 125 which allows the compression strap 140 to exit. In addition, the compression strap 140 may include a first end 150 and a second end 155 that extends out of the slit 145. In or more embodiments, the first end 150 couples to the second end 155.

As further illustrated in FIGS. 1-3, the mobility sock 105 may include a compressed tibia section 160. The compressed tibia section 160 is the section of the leg 100 commonly known as the calf. The compressed tibia section 160 provides gentle pressure to the lower leg to support blood circulation and continued mobility. The compression rate of the compressed tibia section 160 may have a range of between five to thirty mmHg (i.e, millimeters of mercury).

FIG. 4 is a superior view of a patella section of a leg wearing a mobility support sock. As illustrated in FIG. 4, a knee includes a lateral collateral ligament 165 and medial collateral ligament 170. The compression strap 140 is positioned along the respective ligaments and pulled through the slit 145. The first end 150 and the second end 155 are coupled together such that the compression strap 140 is wrapped around the patella section 120, presses against the respective ligaments, and thus providing support. The compression strap 140 may couple using several different means and methods.

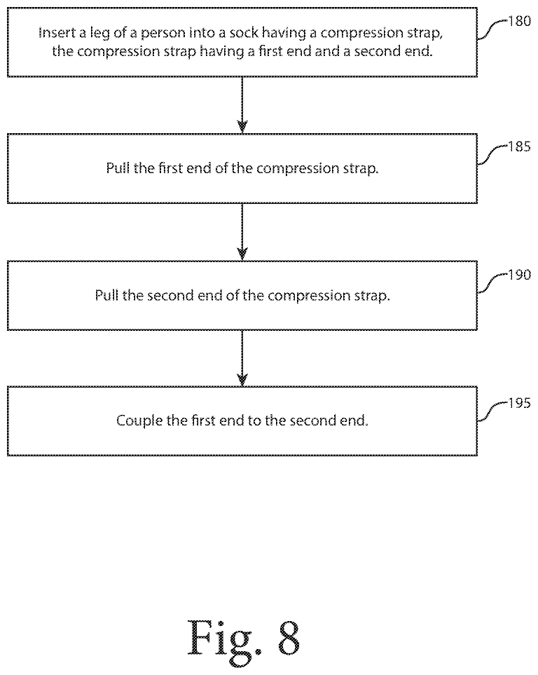

FIG. 5 is an illustration of a compression strap having Velcro straps. FIG. 6 is an illustration of a compression strap having a buckle connection. FIG. 7 is an illustration of a compression strap having a double ring strap. As illustrated in FIG. 5, the compression strap's 140 first end 150 and second end 155 may include Velcro® patches 175 provided by Velcro Industry B.V. or other similar material such that the first end 150 couples to the second end 155. As illustrated in FIG. 7, the compression strap 140 may have a buckle connection. In one or more embodiments, the first end 150 includes a buckle and the second end 155 may include a plurality of notches such that the buckle couples to at least one of the notches. As illustrated in FIG. 8 the compression strap 140 may include double ring set. In one or more embodiments, the first end 150 includes a double ring set for coupling to the second end 155.

FIG. 8 is a flow chart illustrating the method of using a mobility support sock. In operation, a leg (such as leg 100) is inserted into a sock (such as mobility sock 105) having a compression strap (such as compression strap 140), the compression strap (such as compression strap 140) having a first end (such as first end 150) and a second end (such as second end 155) (block 180). The first end (such as first end 150) of the compression strap (such as compression strap 140) is pulled (block 185). The second end (such as second end 155) of the compression strap (such as compression strap 14) is pulled (block 190). The first end (such as first end 150) is coupled to the second end (such as second end 155) (block 195).

In one aspect, the apparatus includes a sock having a femur section and a patella section. The patella section has an outer layer, an inner layer, and a slit on the outer layer. The sock includes a compressed tibia section. The sock includes a compression strap positioned between the outer layer and the inner layer. The compression strap has a first end extending through the slit and a second end extending through the slit.

Implementation may include one or more of the following. The first end and the second end may include Velcro patches such that the first end couples to the second end. The first end may include a double ring set. The femur section may be compressed. The inner layer may include a sip seam coupling the first layer to the second layer such that the compression strap travels along the slip seam. The femur section may include a suspender strap.

In one aspect, the method includes inserting a leg of a person into a sock. The sock has a femur section and a patella section. The patella section has an outer layer, an inner layer, and a slit on the outer layer. The sock includes a compressed tibia section. The sock includes a compression strap positioned between the outer layer and the inner layer. The compression strap has a first end extending through the slit and a second end extending through the slit. The first end of the compression strap is pulled. The second end of the compression strap is pulled. The first end is coupled to the second end.

Implementation may include one or more of the following. The first end and the second end may include Velcro patches such that the first end couples to the second end. The first end may include a double ring set. The femur section may be compressed. The inner layer may include a sip seam coupling the first layer to the second layer such that the compression strap travels along the slip seam. The femur section may include a suspender strap.

The operations of the flow diagrams are described with references to the systems/apparatus shown in the block diagrams. However, it should be understood that the operations of the flow diagrams could be performed by embodiments of systems and apparatus other than those discussed with reference to the block diagrams, and embodiments discussed with reference to the systems/apparatus could perform operations different than those discussed with reference to the flow diagrams.

The word “coupled” herein means a direct connection or an indirect connection.

The text above describes one or more specific embodiments of a broader invention. The invention also is carried out in a variety of alternate embodiments and thus is not limited to those described here. The foregoing description of an embodiment of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. An apparatus for supporting a lower leg of a person comprising:
a sock having:
a femur section;
a patella section, having:
an outer layer,
an inner layer; and
a slit on the outer layer; and
a compressed tibia section;
a compression strap positioned between the outer layer and the inner layer, the compression strap having a first end extending through the slit and a second end extending through the slit.

2. The apparatus of claim 1 wherein the first end and the second end include Velcro patches such that the first end couples to the second end.

3. The apparatus of claim 1 wherein:
the first end includes a buckle; and
the second end includes a plurality of notches such that the buckle couples to at least one of the notches.

4. The apparatus of claim 1 wherein the first end includes a double ring set.

5. The apparatus of claim 1 wherein the femur section is compressed.

6. The apparatus of claim 1 wherein the inner layer includes a slip seam coupling the first layer to the second layer such that the compression strap travels along the slip seam.

7. The apparatus of claim 1 wherein the femur section includes a suspender strap.

8. The apparatus of claim 1 wherein the sock is comprised of nylon.

9. The apparatus of claim 1 wherein the sock is comprised of cotton.

10. A method for supporting a lower leg of a person comprising:
inserting a leg of a person into a sock, the sock having:
a femur section;
a patella section, having:
an outer layer,
an inner layer; and
a slit on the outer layer; and
a compressed tibia section;
a compression strap positioned between the outer layer and the inner layer, the compression strap having a first end extending through the slit and a second end extending through the slit;
pulling the first end of the compression strap;
pulling the second end of the compression strap; and
coupling the first end to the second end.

11. The method of claim 10 wherein the first end and the second end include Velcro patches such that the first end couples to the second end.

12. The method of claim 10 wherein:
the first end includes a buckle; and
the second end includes a plurality of notches such that the buckle couples to at least one of the notches.

13. The method of claim 10 wherein the first end includes a double ring set.

14. The method of claim 10 wherein the femur section is compressed.

15. The method of claim 10 wherein the inner layer includes a slip seam coupling the first layer to the second layer such that the compression strap travels along the slip seam.

16. The method of claim 10 wherein the femur section includes a suspender strap.

17. The method of claim 10 wherein the sock is comprised of nylon.

18. The method of claim 10 wherein the sock is comprised of cotton.
